(19) United States
(12) Patent Application Publication
(10) Pub. No.: US 2013/0183677 A1
(43) Pub. Date: Jul. 18, 2013

(54) METHOD FOR PREDICTING DIFFERENTIATION-INDUCING PROPERTIES TO REGULATORY T-CELLS, BIOMARKER USED FOR THE METHOD, AND USE THEREOF

(71) Applicant: SYSMEX CORPORATION, Kobe-shi (JP)

(72) Inventors: Masatoshi YANAGIDA, Kobe-shi (JP); Masafumi IKEDA, Akashi-shi (JP); Hitoshi UGA, Kobe-shi (JP); Hirokazu KURATA, Kobe-shi (JP)

(73) Assignee: SYSMEX CORPORATION, Kobe-shi (JP)

(21) Appl. No.: 13/743,041

(22) Filed: Jan. 16, 2013

(30) Foreign Application Priority Data
Jan. 17, 2012 (JP) 2012-007389

(51) Int. Cl. C12Q 1/48

(52) U.S. Cl. CPC C12Q 1/485
USPC 435/6.12; 435/15; 536/23.2; 435/194

(57) ABSTRACT

A method for predicting differentiation-inducing properties of naive T-cells to regulatory T-cells comprising: measuring an amount of ZAK in naive T-cells contained in the body fluid collected from the living body; and predicting differentiation-inducing properties of the naive T-cells to regulatory T-cells based on the measurement results is disclosed. A method for determining the risk of development of Graft versus Host Disease and a biomarker for predicting differentiation-inducing properties of naive T-cells to regulatory T-cells are also disclosed.

### FIELD OF THE INVENTION

The present invention relates to a method for predicting differentiation-inducing properties of naive T-cells in the body fluid collected from the living body to regulatory T-cells (hereinafter referred to as “Treg cells”). Further, the present invention relates to a method for determining the risk of development of Graft versus Host Disease (GVHD) when the body fluid collected from the living body is used as a transplant. The present invention further relates to a biomarker for predicting differentiation-inducing properties of naive T-cells to Treg cells.

### BACKGROUND

Currently, hematopoietic stem cell transplantation is widely used as an effective therapy for hematologic malignancy such as leukemia and malignant lymphoma and diseases such as severe aplastic anemia and congenital immunodeficiency. Hematopoietic stem cells differentiate into blood cells in the body of a patient to increase the number of normal blood cells. Thus, an immunity to the disease is provided to the patient.

However, one of the problems of hematopoietic stem cell transplantation includes GVHD. GVHD is the general term for symptoms developed when the immune system of donor-derived immunocytes (mainly T-cells) contained in a transplant recognizes the patient's body as foreign and attack it. Severe symptoms may result in death.

There have been many reports on the association between patients with transplanted hematopoietic stem cells and GVHD. For example, the reference (J. M. Magenau et al., Biol Blood Marrow Transplant. 2010 July; 16 (7): 907-914) discloses that the number of Treg cells in the peripheral blood is decreased in a linear manner as GVHD in bone marrow transplantation patients becomes severe. The reference describes that the Treg cells may be biomarkers for diagnosing and predicting GVHD.

The reference (K. L. Hippen et al., Am. J Transplant. 2011 June; 11 (6): 1148-1157) discloses that the Treg cells differentiated and induced from naive T-cells from human peripheral blood reduce the case fatality rate of GVHD model mice.

Recently, cord blood transplant has attracted attention as a method for transplanting hematopoietic stem cells. The impact on donors at the time of cord blood (CB) collection is minimal and a large amount of hematopoietic stem cells is contained therein. Accordingly, the cord blood is easily used as a source of hematopoietic stem cells. It is considered that the absolute number of T-cells in the cord blood is small and the risk of development of GVHD is lower.

Further, it is considered that a difference in characteristics between naive T-cells contained in cord blood and naive T-cells in adult peripheral blood contributes a low level of the risk of development of GVHD in the cord blood transplant. For example, the reference (J. H. Lee et al., J Immunol. 2011 Aug. 15; 187 (4): 1778-1787) discloses that naive T-cells in cord blood likely differentiate and induce into Treg cells as compared with naive T-cells in adult peripheral blood.

As described above, the cord blood transplant shows promise for the procedure of hematopoietic stem cell transplantation which is less likely to develop GVHD. However, it is known that it may cause GVHD. It is contemplated that if differentiation-inducing properties of naive T-cells contained in the body fluid such as cord blood to Treg cells can be predicted, the risk of development of GVHD caused by a transplant such as cord blood can be previously determined. Accordingly, there is a demand for providing a method for predicting differentiation-inducing properties of naive T-cells contained in the body fluid to Treg cells.

### SUMMARY OF THE INVENTION

The scope of the present invention is defined solely by the appended claims, and is not affected to any degree by the statements within this summary.

In order to solve the above problems, the present inventors have conducted intensive examinations. As a result, they have found that if naive T-cells having a high expression level of sterile alpha motif and leucine zipper containing kinase AZK (ZAK) gene are cultured under conditions suitable for differentiation induction into Treg cells, the expression level of the FOXP3 gene in the cultured cells is increased. Here, it is known that the FOXP3 is a transcription factor involved in the differentiation into Treg cells and is also a marker specific to Treg cells. That is, they have found that naive T-cells having a high expression level of ZAK tend to have high differentiation-inducing properties to Treg cells. Thus, the present invention has been completed.

According to a first aspect of the present invention, a method for predicting differentiation-inducing properties of naive T-cells to regulatory T-cells comprising:

measuring an amount of ZAK in naive T-cells contained in the body fluid collected from the living body; and

predicting differentiation-inducing properties of the naive T-cells to regulatory T-cells based on the measurement results.

According to a second aspect of the present invention, a method for determining the risk of development of Graft versus Host Disease comprising:

measuring the amount of ZAK in naive T-cells contained in the body fluid collected from the living body; and

determining the risk of development of Graft versus Host Disease when the body fluid is used as a transplant based on the measurement results.

According to a third aspect of the present invention, a biomarker for predicting differentiation-inducing properties of naive T-cells to regulatory T-cells, comprising a polynucleotide having a base sequence of ZAK gene or a polypeptide having an amino acid sequence of ZAK protein.

### BRIEF DESCRIPTION OF THE DRAWINGS

FIGS. 1A and 1B are graphs showing that CB is classified into two groups based on the expression level of the ZAK gene in CB naive T-cells on the 0th day after collection and the expression level of the FOXP3 gene in the cells obtained by culturing naive T-cells under Treg cell differentiation-inducing conditions (on the 6th day after the start of culture of the cells); and

FIG. 2 is a graph showing a correlation between the expression level of the ZAK gene in CB naive T-cells on the 0th day after collection and the expression level of the FOXP3 gene in the cells obtained by culturing naive T-cells under Treg cell differentiation-inducing conditions (on the 6th day after the start of culture of the cells).

### DETAILED DESCRIPTION OF THE PREFERRED EMBODIMENTS

The method for predicting differentiation-inducing properties of naive T-cells to Treg cells of the present invention (hereinafter simply referred to as “prediction method”) includes measuring an amount of ZAK in naive T-cells contained in the body fluid collected from the living body (measuring process); and predicting differentiation-inducing properties of the naive T-cells to Treg cells based on the measurement results.

In the present invention, the Treg cells are not particularly limited as long as they are cells which are differentiated and induced from naive T-cells, and are involved in the mechanism of immune tolerance. Examples of the Treg cells include FOXP3 positive cells.

In the embodiment of the present invention, the body fluid is not particularly limited as long as it is a body fluid which is collected from the living body and contains naive T-cells. Examples thereof include peripheral blood, bone marrow fluid, and cord blood. Among them, cord blood is preferred. The living body from which the body fluid is collected is not particularly limited as long as it is a mammal. Preferably, it is a human.

The amount of ZAK can be measured using any known method in the art and a naive T-cell sample isolated from the body fluid. The isolation of naive T-cells from the body fluid is performed, for example, as follows.

The body fluid collected from the living body is centrifuged to obtain a cell fraction. Then, CD4 positive cells are roughly purified from the fraction using magnetic beads to which anti-CD4 antibodies are bound.

The CD4 positive cells thus obtained are stained using fluorescence-labeled antibodies. Thereafter, cells of CD4⁺CD25⁻CD45RA⁺CD45RO⁻ are separated as naive T-cells (hereinafter simply referred to as “naive T-cells”) using a cell sorter.

In the measuring process, the ZAK being measured is known to be a molecule belonging to the MAPKKK family. It is known that the ZAK protein encoded by the ZAK gene is activated by phosphorylation, and the activated ZAK protein phosphorylates the MMK7 protein.

The base sequence of the ZAK gene itself is already known. These can be known by, for example, UniGene (database provided by National Center for Biotechnology Information: NCBI). The base sequence of the ZAK gene and the amino acid sequence of the ZAK protein (Entrez gene ID, UniGene ID, Transcript ID, Protein ID, and Affymetrix Probe SetID) are shown in Table 1.

TABLE 1

| | Entrez | | Transcript | | Affymetrix |
|---|---|---|---|---|---|
| Gene Symbol | Gene ID | UniGeneID | ID | Protein ID | Probe Set ID |
| ZAK | 51776 | Hs.444451 | NM_016653.2 | NP_057737.2 | 1555259_at |
| | | | (SEQ ID | (SEQ ID | 218833_at |
| | | | NO. 1) | NO. 3) | 222757_s_at |
| | | | NM_133646.2 | NP_598407.2 | 223519_at |
| | | | (SEQ ID | (SEQ ID | 225662_at |
| | | | NO. 2) | NO. 4) | 225665_at |
| | | | | | 238613_at |

The term “amount of ZAK” used herein means both of “the expression level of ZAK” and “the activity of ZAK”. Here, “the expression level of ZAK” means both of “the expression level of ZAK” and “the expression level of the ZAK protein”. Further, “the activity of ZAK” can be represented by “the activity value of ZAK”. “The activity value of ZAK” means a value showing the degree of the activity represented by the function of the ZAK protein. Examples of the activity value include kinase activity values.

The term “value showing the amount of ZAK” used herein may be a measured value itself or a value calculated based on the value and it can be represented by any form or unit, such as mass (weight), concentration, ratio, intensity or level.

The term “expression level of the ZAK gene” means an amount of mRNA of the ZAK gene or an amount of material reflecting the amount, e.g., the amount of cDNA or cRNA synthesized from the mRNA.

The expression level of the ZAK gene can be measured, for example, as follows.

First, a nucleic acid (RNA) is extracted from a sample containing naive T-cells by any known method in the art, such as phenol extraction or ethanol precipitation. Then, the expression level of the ZAK gene in the obtained nucleic acid is measured. The nucleic acid may be extracted using a commercially available RNA extraction kit.

The expression level of the ZAK gene can be measured by any known method in the art, such as a nucleic acid amplification technique (e.g., Quantitative RT-PCR assay and Loop-mediated isothermal amplification (LAMP)), a hybridization technique (e.g., Northern hybridization) or microarray analysis. The primers and nucleic acid probes to be used in these techniques can be produced by any known method in the art. The base sequences thereof can be suitably determined based on the base sequence of the ZAK gene.

The term “expression level of the ZAK protein” used herein means an amount of the protein encoded by the ZAK gene.

The expression level of the ZAK protein can be measured, for example, as follows.

First, the protein is extracted from cells. The extraction of the protein from cells can be performed by any known method, such as ultrasonic breaking of cells or solubilization using cell lysates. Then, an antibody specifically bound to the ZAK protein is used so that the ZAK protein can be measured. Specifically, the ZAK protein can be measured by any known method in the art, such as Enzyme-linked immunosorbent assay (ELISA), Western blotting or Lowry method.

The antibody can be produced by, for example, a known procedure as follows. Based on the base sequence of the ZAK gene or the amino acid sequence of the ZAK protein, a DNA molecule encoding a protein having an amino acid sequence of ZAK protein is incorporated into a suitable expression vector. The obtained expression vector is introduced into a suitable host cell. The obtained transformed cells are cultured to produce the ZAK protein. The obtained protein is purified and used as an immunogen. Suitable mammals such as rats and mice are immunized using the immunogen and an adjuvant, if desired. An antibody-producing cell which produces an antibody directed to the target immunogen is selected from spleen cells in the immunized animals by screening. The obtained antibody-producing cell is fused with a myeloma cell to obtain a hybridoma. The hybridoma is screened to obtain an antibody-producing hybridoma which produces the antibody which is specifically bound to the protein encoded by the ZAK gene. The obtained antibody-producing hybridoma is cultured to obtain an antibody specifically bound to the ZAK protein.

The term “kinase activity value” used herein means a phosphorylation ability of a certain kinase on its target substance or an amount showing the activity or a value reflecting either of them.

Measurement of the kinase activity value can be performed by measuring the phosphorylated state of a substrate phosphorylated by the ZAK protein using any known measurement method. Examples of the substrate to be used in the measurement include a universal substrate having a low specificity to the ZAK protein kinase and a protein encoded by the gene involved in the MAP kinase cascade to which the ZAK gene belongs (e.g., MMK7 protein). Examples of the measurement method to be used in the measurement include ³²P autoradiography, ELISA, and phosphorylation analysis by mass spectrometry (MS).

The kinase activity value can be measured using a commercially available kit for measuring the kinase activity value. Examples of the measurement kit include ADP-Glo (trademark) Kinase and Max Assay (Promega KK.).

In the prediction method of the present invention, differentiation-inducing properties of naive T-cells to Treg cells are predicted based on the measurement results obtained in the measuring process.

Here, the differentiation-inducing properties mean properties of naive T-cells to differentiate to Treg cells or the degree of activity.

As described above, the present inventors have found that the amount of ZAK in naive T-cells is correlated with the expression level of FOXP3 in cells after culturing the naive T-cells under conditions suitable for differentiation induction into Treg cells.

Therefore, in the present invention, the differentiation-inducing properties of naive T-cells to Treg cells predicted based on the amount of ZAK is said to indicate the expression level of the FOXP3 gene in cells cultured under conditions suitable for differentiation induction or the expression level of the protein encoded by the gene.

In the preferred embodiment of the present invention, differentiation-inducing properties of naive T-cells contained in a transplant to Treg cells are predicted by comparing the value showing the amount of ZAK thus measured with a threshold in the prediction process.

More specifically, differentiation-inducing properties are predicted to be high when the value showing the amount of ZAK is higher than the threshold in the prediction process. On the contrary, differentiation-inducing properties are predicted to be low when the value showing the amount of ZAK is lower than the threshold.

The threshold is not particularly limited and can be experimentally set by the accumulation of data. For example, the threshold may be set as follows. First, some of naive T-cells contained in a plurality of body fluid samples are taken, and the amount of ZAK in the samples is measured. Subsequently, the remaining naive T-cells are cultured under known conditions suitable for differentiation induction into Treg cells. The FOXP3 expression level in the obtained cells is measured. The value showing the amount of ZAK which can clearly classify the FOXP3 expression level in the measured specimens into high and low groups is set as a threshold.

As described above, it is known that Treg cells suppress the development of GVHD after transplantation of hematopoietic stem cells. In the present invention, the amount of ZAK shows correlation with differentiation-inducing properties of naive T-cells to Treg cells. Therefore, the risk of development of GVHD when the body fluid collected from the living body is used as a transplant can be determined based on the amount of ZAK thus obtained.

The method for determining the risk of development of Graft versus Host Disease of the present invention (hereinafter simply referred to as “determination method”) includes measuring the amount of ZAK in naive T-cells contained in the body fluid collected from the living body; and determining the risk of development of Graft versus Host Disease when the body fluid is used as a transplant based on the measurement results.

In the determination method of the present invention, the process of measuring the amount of ZAK can be performed in the same manner as the process described in the method for predicting differentiation-inducing properties of naive T-cells to Treg cells.

In the embodiment of the determination method of the present invention, the risk of development of Graft versus Host Disease when the body fluid is used as a transplant is determined based on the results obtained by comparing the value showing the amount of ZAK with a threshold in the determination process.

More specifically, the risk of development is determined to be low when the value showing the amount of ZAK is higher than the threshold in the determination process. On the contrary, the risk of development is determined to be high when the value showing the amount of ZAK is lower than the threshold.

In the determination process, the amount of ZAK to be used is the same as the defined value. As described above, in the present invention, the amount of ZAK shows correlation with differentiation-inducing properties of naive T-cells to Treg cells. Therefore, the threshold to be used for the prediction method of the present invention may be used as a threshold which can clearly classify the body fluid as a transplant into high and low groups of risk of development of GVHD. Accordingly, in the prediction method of the present invention, when the body fluid containing naive T-cells which is predicted to have high differentiation-inducing properties to Treg cells is used as a transplant, the risk of development of GVHD can be determined to be low. On the contrary, in the prediction method of the present invention, when the body fluid containing naive T-cells which is predicted to have low differentiation-inducing properties to Treg cells is used as a transplant, the risk of development of GVHD can be determined to be high.

The scope of the present invention includes a biomarker for predicting differentiation-inducing properties of naive T-cells to Treg cells, including a polynucleotide having a base sequence of ZAK gene or a polypeptide having an amino acid sequence of ZAK protein (hereafter simply referred to as “biomarker”).

When the biomarker of the present invention includes a polynucleotide having a base sequence of ZAK gene, the amount of the polynucleotide marker can be measured by an arbitrary known measurement method. For example, the amount of the polynucleotide marker can be measured by any known method in the art, such as a nucleic acid amplification technique such as Quantitative RT-PCR or Loop-mediated isothermal amplification (LAMP), a hybridization technique (e.g., Northern hybridization and Fluorescence in situ hybridization (FISH)) or microarray analysis.

When the biomarker of the present invention includes a polypeptide having an amino acid sequence of ZAK protein, the amount of the polypeptide marker can be measured using an arbitrary known measurement method. For example, the ZAK protein can be measured by any known method in the art, such as Enzyme-linked immunosorbent assay (ELISA), Western blotting or Lowry method.

When the biomarker of the present invention includes a polypeptide having an amino acid sequence of ZAK protein, the kinase activity value of ZAK protein can be measured. Measurement of the kinase activity value of ZAK protein can be performed by measuring the phosphorylated state of a substrate phosphorylated by the ZAK protein using any known measurement method. Examples of the substrate to be used in the measurement include a universal substrate having a low specificity to the ZAK protein kinase and a protein encoded by the gene involved in the MAP kinase cascade to which the ZAK gene belongs (e.g., MMK7 protein). Examples of the measurement method to be used in the measurement include ³²P autoradiography, ELISA, and phosphorylation analysis by mass spectrometry (MS).

The kinase activity value can be measured using a commercially available kit for measuring the kinase activity value. Examples of the measurement kit include ADP-Glo (trademark) Kinase and Max Assay (Promega KK.).

The scope of the present invention includes a reagent for predicting differentiation-inducing properties of naive T-cells to Treg cells which contains a primer set or nucleic acid probe for analyzing the expression level of the ZAK gene, an antibody specifically bound to the ZAK protein, or a substrate phosphorylated by the ZAK protein. The reagent of the present invention may further contain the primer set, the nucleic acid probe, at least one labeled molecule for labeling an antibody or a substrate (e.g., ³²P, horseradish peroxidase (HRP)) or the like. The reagent of the present invention may contain reagents such as a buffer solution, a chromophoric substrate, a secondary antibody, and a blocking agent, if necessary.

When the reagent of the present invention contains the primer set for analyzing the expression level of the ZAK gene, the reagent of the present invention can be used to measure the expression level of the ZAK gene and predict differentiation-inducing properties of naive T-cells to Treg cells. In this case, as the method for measuring the expression level of the ZAK gene, an arbitrary known measurement method can be used. Examples thereof include Quantitative RT-PCR and LAMP.

When the reagent of the present invention contains the nucleic acid probe for analyzing the expression level of the ZAK gene, the reagent of the present invention can be used to measure the expression level of the ZAK gene and predict differentiation-inducing properties of naive T-cells to Treg cells. In this case, as the method for measuring the expression level of the ZAK gene, an arbitrary known measurement method can be used. Examples thereof include FISH, Northern hybridization, and microarray analysis.

When the reagent of the present invention contains the antibody specifically bound to the ZAK protein, the reagent of the present invention can be used to measure the expression level of the ZAK protein and predict differentiation-inducing properties of naive T-cells to Treg cells. In this case, as the method for measuring the expression level of the ZAK protein, an arbitrary known measurement method can be used. Examples thereof include ELISA, Western blotting, and Lowry method.

When the reagent of the present invention contains the substrate phosphorylated by the ZAK protein, the reagent of the present invention can be used to measure the kinase activity value of ZAK and predict differentiation-inducing properties of naive T-cells to Treg cells. In this case, measurement of the kinase activity value of ZAK protein can be performed by measuring the phosphorylated state of a substrate phosphorylated by the ZAK protein using any known measurement method. Examples of the substrate to be used in the measurement include a universal substrate having a low specificity to the ZAK protein kinase and a protein encoded by the gene involved in the MAP kinase cascade to which the ZAK gene belongs (e.g., MMK7 protein). Examples of the measurement method to be used in the measurement include ³²P autoradiography, ELISA, and phosphorylation analysis by mass spectrometry (MS).

### EXAMPLES

### Example 1

### 1. Separation of Naive T-Cells

CD4 positive cells were roughly purified from human cord blood (n=13; Institute of Physical and Chemical Research (RIKEN), Bioresource center using magnetic beads having anti-CD4 antibodies bound thereto (Miltenyi Biotec).

The CD4 positive cells thus obtained were stained with fluorescence-labeled antibodies shown in Table 2, followed by screening of CD25-negative CD45RA-positive CD45RO-negative cells using a cell sorter (FACS Aria: Becton Dickinson) to separate the cells of CD4⁺CD25⁻CD45RA⁺CD45RO⁻ as naive T-cells.

TABLE 2

| | Fluorescently-labeled | | |
|---|---|---|---|
| Antigen | substance | Clone | Manufacturer |
| CD4 | FITC | OKT4 | BioLegend, Inc. |
| CD25 | PE-Cy7 | BC96 | eBioscience, Inc. |
| CD45RA | APC | UCHL1 | BioLegend, Inc. |
| CD45RO | PE | HI100 | BioLegend, Inc. |

2. Differentiation Culture of Naive T-Cells into Treg Cells

The CB naive T-cells (n=13) were cultured in the Yssel medium in the presence of 10 ng/ml of IL-2 and 10 ng/ml of TGF-β, for six days to differentiate and induce into Treg cell.

### 3. Measurement of Expression Level of ZAK Gene and Expression Level of FOXP3 Gene

mRNAs were extracted from the separated CB naive T-cells. The expression level of the ZAK gene in the mRNAs on the 0th day after collection was measured by Quantitative RT-PCR using the hZAK-684F primer (5′-ACACACATGTCCTTGGTTGGAA-3′; SEQ ID NO. 5) and the hZAK-753R primer (5′-TGACACAGGGAGACTCTGGATAAC-3′; SEQ ID NO. 6). mRNAs were extracted from the Treg cells after differentiation culture. The expression level of the FOXP3 gene was measured by Quantitative RT-PCR using the hFOXP3_{—}963F primer (5′-CACCTGGCTGGGAAAATGG-3′; SEQ ID NO. 7) and the hFOXP3_{—}1025R primer (5′-GGAGCCCTTGTCGGATGAT-3′; SEQ ID NO. 8).

### 4. Correlation Between Expression Level of ZAK Gene and Expression Level of FOXP3 Gene

The measurement result of the expression level of the ZAK gene in CB naive T-cells are compared with the measurement result of the expression level of the FOXP3 gene in the cells after culture of CB naive T-cells under Treg cell differentiation-inducing conditions to examine a correlation between the expression level of the ZAK gene and the expression level of the FOXP3 gene.

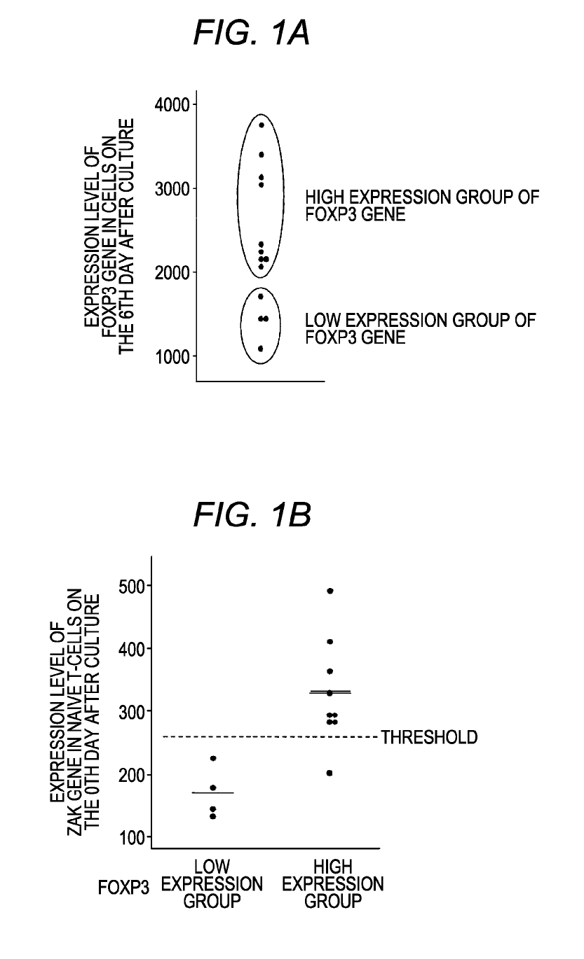

The results are shown in FIG. 1. FIG. 1A shows the expression levels of the FOXP3 gene in the cells cultured under Treg cell differentiation-inducing conditions for six days (n=13). As shown in FIG. 1A, human cord blood (CB) was classified into two groups: low- and high-expression groups of FOXP3 gene. FIG. 1B shows the expression levels of the ZAK gene in CB naive T-cells on the 0th day after collection from human cord blood CB in the groups. As shown in FIG. 1B, the expression levels of the ZAK gene in the low expression group of FOXP3 gene were low, while the expression levels of the ZAK gene in the high expression group of FOXP3 gene were high. As shown in FIG. 1B, the low- and high-expression groups of FOXP3 gene can be distinguished by setting a threshold to the expression level of the ZAK gene. The threshold set in FIG. 1B is an example of settable thresholds and it is not limited thereto.

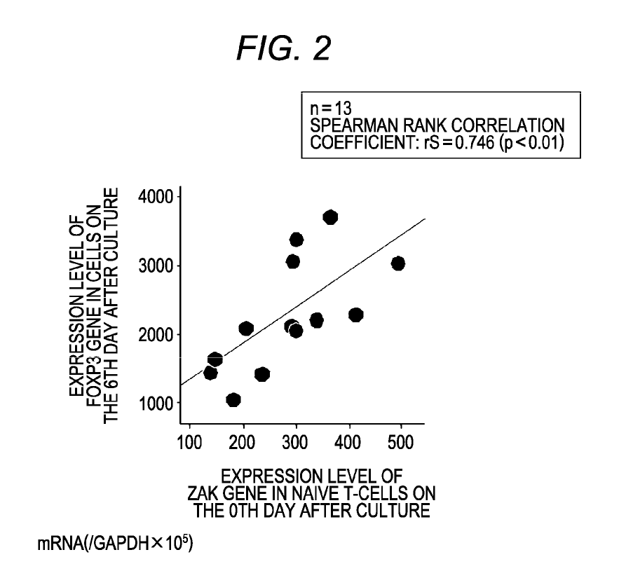

Then, a correlation between the expression level of the ZAK gene and the expression level of the FOXP3 gene in the cells cultured under Treg cell differentiation-inducing conditions for six days was examined. The results are shown in FIG. 2. FIG. 2 showed that the expression level of the ZAK gene in CB naive T-cells on the 0th day after collection was significantly correlated with the expression level of the FOXP3 gene in the cells cultured under Treg cell differentiation-inducing conditions for six days.

1. A method for predicting differentiation-inducing properties of naive T-cells to regulatory T-cells comprising:
measuring an amount of ZAK in naive T-cells contained in the body fluid collected from the living body; and
predicting differentiation-inducing properties of the naive T-cells to regulatory T-cells based on the measurement results.

2. The method according to claim 1, wherein the differentiation-inducing properties are predicted based on the results obtained by comparing a value showing the amount of ZAK with a threshold in the prediction process.

3. The method according to claim 2, wherein the differentiation-inducing properties are predicted to be high when the value showing the amount of ZAK is higher than the threshold in the prediction process.

4. The method according to claim 2, wherein the differentiation-inducing properties are predicted to be low when the value showing the amount of ZAK is lower than the threshold in the prediction process.

5. The method according to claim 1, wherein the body fluid is cord blood, bone marrow fluid or peripheral blood.

6. The method according to claim 1, wherein the amount of ZAK is an expression level of ZAK.

7. The method according to claim 6, wherein the expression level of ZAK is an expression level of ZAK gene or an expression level of ZAK protein.

8. The method according to claim 2, wherein the value showing the amount of ZAK is an activity value of ZAK.

9. The method according to claim 8, wherein the activity value of ZAK is a kinase activity value of ZAK protein.

10. The method according to claim 1, wherein the regulatory T-cells are FOXP3 positive cells.

11. A method for determining the risk of development of Graft versus Host Disease comprising:
measuring the amount of ZAK in naive T-cells contained in the body fluid collected from the living body; and
determining the risk of development of Graft versus Host Disease when the body fluid is used as a transplant based on the measurement results.

12. The method according to claim 11, wherein the risk of development is determined based on the results obtained by comparing a value showing the amount of ZAK with a threshold in the determination process.

13. The method according to claim 12, wherein the risk of development is predicted to be low when the value showing the amount of ZAK is higher than the threshold in the determination process.

14. The method according to claim 12, wherein the risk of development is predicted to be high when the value showing the amount of ZAK is lower than the threshold in the determination process.

15. The method according to claim 11, wherein the body fluid is cord blood, bone marrow fluid or peripheral blood.

16. The method according to claim 11, wherein the amount of ZAK is an expression level of ZAK.

17. The method according to claim 16, wherein the expression level of ZAK is an expression level of ZAK gene or an expression level of ZAK protein.

18. The method according to claim 12, wherein the value showing the amount of ZAK is an activity value of ZAK.

19. The method according to claim 18, wherein the activity value of ZAK is a kinase activity value of ZAK protein.

20. A biomarker for predicting differentiation-inducing properties of naive T-cells to regulatory T-cells, comprising a polynucleotide having a base sequence of ZAK gene or a polypeptide having an amino acid sequence of ZAK protein.


SEQUENCE LISTING (8 sequences)

SEQ ID NO: 1 - RNA, 3867 nt, Homo sapiens
acugucuuccucauuggcgccgugcagagaggcggaauguucaacuccuaacugcggcggaaacgugggagccgcgcgggccgcugucgucccaacccccgccgcccucgucgcgcgcggggccuccgcgcccccggcugcugcucacgccccgcccgggagccagauuuuguggaaguauaauacuuugucauuaugagaugucgucucucggugccuccuuugugcaaauuaaauuugaugacuugcaguuuuuugaaaacugcgguggaggaaguuuugggaguguuuaucgagccaaauggauaucacaggacaaggagguggcuguaaagaagcuccucaaaauagagaaagaggcagaaauacucaguguccucagucacagaaacaucauccaguuuuauggaguaauucuugaaccucccaacuauggcauugucacagaauaugcuucucugggaucacucuaugauuacauuaacaguaacagaagugaggagauggauauggaucacauuaugaccugggccacugauguagccaaaggaaugcauuauuuacauauggaggcuccugucaaggugauucacagagaccucaagucaagaaacguuguuauagcugcugauggaguauugaagaucugugacuuuggugccucucgguuccauaaccauacaacacacauguccuugguuggaacuuucccauggauggcuccagaaguuauccagagucucccugugucagaaacuugugacacauauuccuauggugugguucucugggagaugcuaacaagggagguccccuuuaaagguuuggaaggauuacaaguagcuuggcuuguaguggaaaaaaacgagagauuaaccauuccaagcaguugccccagaaguuuugcugaacuguuacaucaguguugggaagcugaugccaagaaacggccaucauucaagcaaaucauuucaauccuggaguccaugucaaaugacacgagccuuccugacaaguguaacucauuccuacacaacaaggcggaguggaggugcgaaauugaggcaacucuugagaggcuaaagaaacuagagcgugaucucagcuuuaaggagcaggagcuuaaagaacgagaaagacguuuaaagaugugggagcaaaagcugacagagcaguccaacaccccgcugcugccuuccuuugagauuggugcauggacggaagacgauguguauuguuggguucagcagcucgucagaaaaggugacucuucagcagagaugaguguauaugcaagcuuguuuaaagaaaacaacauuacagggaagcggcugcugcugcuggaggaagaagaccugaaagacaugggcauugucuccaaggggcauaucauucacuucaagucagccauugagaaauuaacccaugauuacauaaauuuguuucacuucccaccacuaauuaaggacucaggaggugaaccugaagaaaaugaggaaaaaauagugaaccuggaacugguuuuugguuuucacuugaaaccaggaacuggcccacaggauuguaaguggaaaauguauauggagauggauggggaugaaauugcaauaaccuacauaaaagaugugacauucaacacuaaccuaccugaugcggagauuuuaaagaugacaaagccaccauuuguaauggagaaguggauuguaggaauagcaaaaagucagacuguggagugcacugucacauaugagagugauguuagaacuccaaaaagcacuaaacauguccauucgauucaguggaguagaacaaaaccucaggaugaagugaaagcaguccaacuugccauucagacauuauucaccaauucagauggcaacccuggaagcagguccgacucaagugcugauugccagugguuagauacucugaggaugcggcagauugcauccaacacuucuuuacagcguucccagagcaauccuauucuggggucaccguucuucucacacuuugauggccaggauuccuacgcugcugcugugagacggccccaggugcccauuaaguaucaacagauuacaccugugaaccaguccagaagcucgucuccuacucaguauggacugaccaaaaacuucucuucccuacaucucaacucuagggacaguggcuuuuccaguggcaauacugacaccucuucagagaggggucgauacucagacagaagcaggaacaaauauggacgugguaguauaucacucaauucuucuccuagaggaagauacaguggaaagagucagcauuccacuccuucaagaggaagauacccuggaaaguucuacaggguuucucagucagcacucaauccucaccagucgccugacuucaagagaagccccagggaccuccaccaacccaacaccauaccagggaugccuuugcacccugagacugacucaagagccagugaagaggacagcaaagucagcgaagggggcuggacaaaaguggaauaccggaaaaagccccacaggccaucucccgccaaaaccaauaaagagagagccagaggggaccaccguggauggagaaacuuuugaugaauugaacuacauagcuuuucuaagcagguuaaaaaaaaaaaaaaaaagaaauguaaugguuuuugauaauaugaucccuucagauugaauuaacgaaaagacaacacuuccaguuuuuggauugggaaauaccuucuaauugagacuauagccaaaccagggccaaaauuauggauauuggucacccagugaucauaacuaggcuugaaaaucacuacacauauuuucugccuugagugaacauuuuuagaggaaagguuaugccaucuuuuuacccuaaccacugauauucugguuagcagggccaggacaaggggaaggaaaaugaggucaacaaaaaaaucaaauuuuuaggaaaagauaagaugaauguuacugauuuuuccuuuuggcugaggcugcaauauggccuggcaaggcacuguuacugaucuugucuuuaacauuuugauauuuuguucaucauaauuuuugcauuuauuuuuuuaaauauugcauuaaaauaucauuuagcuugauuaucgaguuuuuugguuugagguuuuuuguugcuucuuuuuucuuuucuuucuuucccccucuuuuuuuuggauguccccuuaaauuuugugcccaaggcagguaccucacucaucucauccuuggcucagcccugcugguuaguauuuaguauuuauuuuaguaagauauuugugucuguaugauggucagaguugaacugaucuggcuugucauuuuucaguaauaaaaaaaguuacugaauuuaauguugaauaugaugcauaucucauucauuacgauuuaucagaaaccaaagauuuaaauugccuagauuugugguucuuucucuuccuaaguucccagcgacugcuuucaaauacuauuuucuaaauuucaccaaaggagcaaagaggauaaaacaacacuccauaaaggccucuugggaugucagaaaucuaaaaucuaaaagaaaacagacacagagcaagacaauaacaucacaagcuaaaagccagagaaauuuaaaauuaccaacauccuuguuggaguaagacaguaaauaucagccuugcagcaagacagcucugagcagcugugggcaaagagguaaaccagugggggugcaaggagacugucugcagcuuagggcagaaauggugggauccaacuugugaaaugcuucauguuuuacaaaccaaaaagucagguagcaacaaacuuauuguaugucaaaucaauaaauguuacuuucaauaaaaaa

SEQ ID NO: 2 - RNA, 7194 nt, Homo sapiens
acugucuuccucauuggcgccgugcagagaggcggaauguucaacuccuaacugcggcggaaacgugggagccgcgcgggccgcugucgucccaacccccgccgcccucgucgcgcgcggggccuccgcgcccccggcugcugcucacgccccgcccgggagccagauuuuguggaaguauaauacuuugucauuaugagaugucgucucucggugccuccuuugugcaaauuaaauuugaugacuugcaguuuuuugaaaacugcgguggaggaaguuuugggaguguuuaucgagccaaauggauaucacaggacaaggagguggcuguaaagaagcuccucaaaauagagaaagaggcagaaauacucaguguccucagucacagaaacaucauccaguuuuauggaguaauucuugaaccucccaacuauggcauugucacagaauaugcuucucugggaucacucuaugauuacauuaacaguaacagaagugaggagauggauauggaucacauuaugaccugggccacugauguagccaaaggaaugcauuauuuacauauggaggcuccugucaaggugauucacagagaccucaagucaagaaacguuguuauagcugcugauggaguauugaagaucugugacuuuggugccucucgguuccauaaccauacaacacacauguccuugguuggaacuuucccauggauggcuccagaaguuauccagagucucccugugucagaaacuugugacacauauuccuauggugugguucucugggagaugcuaacaagggagguccccuuuaaagguuuggaaggauuacaaguagcuuggcuuguaguggaaaaaaacgagagauuaaccauuccaagcaguugccccagaaguuuugcugaacuguuacaucaguguugggaagcugaugccaagaaacggccaucauucaagcaaaucauuucaauccuggaguccaugucaaaugacacgagccuuccugacaaguguaacucauuccuacacaacaaggcggaguggaggugcgaaauugaggcaacucuugagaggcuaaagaaacuagagcgugaucucagcuuuaaggagcaggagcuuaaagaacgagaaagacguuuaaagaugugggagcaaaagcugacagagcaguccaacaccccgcuucucuugccucuugcugcaagaaugucugaggagucuuacuuugaaucuaaaacagaggagucaaacagugcagagaugucaugucagaucacagcaacaaguaacggggagggccauggcaugaacccaagucugcaggccaugaugcugaugggcuuuggggauaucuucucaaugaacaaagcaggagcugugaugcauucugggaugcagauaaacaugcaagccaagcagaauucuuccaaaaccacaucuaagagaagggggaagaaagucaacauggcucugggguucagugauuuugacuugucagaaggugacgaugaugaugaugaugacggugaggaggaggauaaugacauggauaauagugaaugaaagcagaaagcaaaguaauaaaaucacaaauguuuggaaaacacaaaaguaacuuguuuaucucagucuguacaaaaacaguaaggaggcagaaagccaagcacugcauuuuuaggccaaucacauuuacaugaccguaauuucuuaucaauucuacuuuuauuuuugcuuacagaaaaacggggggagaauuaagccaaagaaguauauuuaugaaucagcaaauguggugccugauuauagaaauuugugauccuauauacaauauaggacuuuuaaaguugugacauucuggcuuuuucuuuuaaugaauacuuuuuaguuuguauuugacuuuauuuccuuuauucaaaucauuuuuaaaaacuuacauuuugaacaaacacucuuaacuccuaauuguucuuugacacguaguaauucugugacauacuuuuuuuuucuuauagcaauacacuguaauaucagaaaugguuggccugagcaaccuaguaagaccucgucucuacuaauaauuaaaaaacuagcuggcaugguagcacacaccuguagucccagauacuugggaggccaaggcaggaggauugcuugagaccuagcaaucagucagggcugcagugagccaugauggcaccacugcacucuagccugggcaagagaacaagauccugucucaaaaaacaaaaaaaagaaagaauugauaguacaaaauccaacaacaauacugagaugaucuaagaagguuauaacaaaaugcucuucagaaauaccuaagugcugagaauuuuuaguacuaaagagcacagcugcucaaaguaaagccugagcaguguucucaguaauguauuugaaggaaaaauacccugauuugaaaccaacagcagauguugcaaacuuucauaccacugcuggccauggaagccucuuaacaacacacugucauuuaaggcugugcuugugcuuuauacaaagagaaagagguggucuuaaggggaugcuuccaggggggugaguucaugccucuccuguauuuuccagcaagugggguauguguggugguuuguuuuuuagaggggcauaauaauccaggauucuaagcauaugcucagcuauuuuaaagaggaaauuaaauauuauaaaagaaauaguaaagauaaguuauccucacuuaggcaaaagcacagguccuuuccauaucaaguuuagccuaccaggguuguuuuuuguuuuaacccugcuuaauaauguugguguuuuagaaguagauacaggcacugcucugaaaaccuggcuagccaaggauauucucagaauguuaucaccuguuugucaaagcuuguuuaaauuauaaaacacuuuuaauuauauauaugaggcaaaagaacuaagacuuuuuucaaacuaaauuagaaaggagugucauuauuugacuguuaaaccaaaauauuuuuggugggucuuuuuauggaaguuuaaagaaaggacaucaucauagauaugaucuaacaguauuucuaacuauauuugaucauuaaaagccucuuggaauuugaagcgugacguguuucuaaugccccuugagaggugaaaaauaccacauaaugaucaguaugcugugccagcuucauuuggggagaaauaacuaguagaaaguucugggugugagguguacagcagucuagguggcauagugaugaagaaagggaucagagucugacugucacucagaauccugggcucaguugcuugacaaccuugggaaaauuguuuuaucuuugugcgucuguuugcugaucuucagcgugggaauaauaacaguaccuacuugaaaggaucauugugcggauuaaaagaaauaauauauguaaagcacuuuaacacagcaccaggcccacggaaaguggcuaauguuagcuacuaugaauggugccagugaagacacugaaaaauaagugauuucaguaaccuucuggaaagcuaucaguuucaaauaauauuuucucuguaguaugagaugaaauuaaaaguggauagcuuucaggaaagauaaagagaacaugcuuagaauguaagcuaaacagauuuuuucuguugcucuuugaaaacuaugagcccuggccagcuuaaccuggucugaggugagacuaaacacaaaaacaguagauaaaucucucccuaaaagauggauucccccacauacccaugcuacuaguuucucugucuauucacacauauguacaaauacaugaacacagccugucugugcucagacauagagaaguacuaccugacuugagucaaugcacccaagaagaaaagcuuggaguagagcagaagggagggcuugggacuccugucuuuccagcaugcccuggggugcaguggucagccaccugaagagagagccaauagcaugggguuuacaaggcaaagauagucauucauucaacacauauucauagagcuccuucucugugccagacacuguucuggaagauagcuagaugaaaaucuuugcacucacagagcuuacaugccagugagugaagaucgaugauaaauaaagcaaaugcaucauauguucacauuugauaaguauaugccaaaaaaugaagccgggaaggaggacaaggcccauggguggguguugagguuuuuaaaguguggucaggaaaggccccacugauaagguaacauuugagcaagucugaaaaaggcaaggggaucuuuggggcuaacuucgggaucccugcacuuuauguaagaauguaaaccuggagucucauuuaagaaugaucagcaauacguuuagaacauaugaacugaaugaaauggacauuuuuucuuaauuuauguauaaauccauaugauuauacauaaaguucugaugcauuaauaaaagcagccaaauagggccaaagagaaaaauaacaggacucuguacuggaccuaacuuuaucauuaauuagguaauauuuuccucauuucuuuacugcugccauuuuccucaccaguauuccagagauggucauagcucauuacucuaccaccaagaaccuaaaaggaauuagaauacagcagaauuggccucagugaagagcuuaaaauuguucuccucguagaacuggacuauugaucauuaccacgugacguuggcucuauuacuuucuguucccaauguccuucuagugguuugaaaauguuaaaacaucccuaaaaucuaaaucauauaaucagaauucuauagugucccacucuaucuguaaagaucauuuggaagacuuuagacucuauuaauuuuaaaaggaauauuuauuagccauaugcagaauuucuaaugaugauauuguacagcuucuaauucacuuuucagaucaguguuugaaauggcaauuaucaguguuggauuuaguuccaacuacuugauuuacaaaaauguacauuuagagguuaaaagaaacagugagaaauguaaacauucaaaaugauaauugaaucucucaguugugggaauaauuaucagagacaugcaacugaaaaugucucaccuuucaucuuuuuuucuuaauucauaaaguuaucuuguagaauuugaugagacccucccuagucauucucaacuggggcggugcugucaccgaaugguguuugagaguguuggggcuagggcacauuuuugguugucacagcaacugggguggcauuugcugcccagugccaggaauaguaacauuaugaaugccagggacagugugcucaguaaagucuuccauccaaaaggggcagggcacgguggcucacgccuguaaucccagcacuuugggaggccaaggugggcggaucaccugaugucagggguucgagaccagccuggccaacauggugaaacccuguugcuacuaaaaauacaaaaauuggcuggguguggugucacaugccaguaaccccagcuacuagggaggcugaggcaggagaaucacuugaacccgggaggcagagguugcagugagcugagauugcaccacuacacuccagccuggaugacagagugagacuucaucucaaaaaaaaaaaaaaacaaaaaaaaacaacccgguagcauugucccuuccccacugacaaacuuaucaaauccagaagcuuuagaguuucgucucuaauuauuuuucuccugaacaaaauuacccaagucaaaacaaaauguauuuuuagaauuacggcagcauacgaccugaauuuugugaguuucguggcuuuaucuuaaaucaccauuucccuaaaaaugguuucuuucuccuuagaaaugcugguggcaacuugaugaaacagccaaaugcaccagggcaggucacuuucccauuacacugauuccacaauuaaaaaaaaaaaagaaaaaaaacucauugagauagcuacaguucuauagguuaauuuaaagccuccuuuuucuacucauuuuugaaagcaaaauuacauuuuacuauuuuacauaaccagugaaaagacguugaaagccuacagcucacuguuuuuggugcucuggaaauguugaggguggguuuuuaaccagugauuuuuaacgugcagugaauuuguuagacuuuuaaacaccagcuaagguagucaaacuugauccccauuaaaaaucaaggaauuaggggucgggggaggguuuaggagugauccagaaugaccucccagaauuacugugcguacaacuuuauuuuucagaguuuucauuggaaugguaagaguuuuaugaaagacaguuuuaaaacuuauucugaguuaaauauuaauacuuuaaaaaauuauuguacuagacuuauugcagccuuuugaaaguagcagaguuucaucauaccacauauauaacagagcauaaauuuucuauaaucaggcaccuuuugcugcuuuugaguaagacuguuuuccuguuuaaguguuaagcaucgccagacauaaaaaucuauucucuccucucgauuguagcauagccugacagcucuagauacagcauuucuaugaugaaaaaugaguauccaucaggaaaucuagaagacuagccguguuuucucagacuccaccuuuguuugcacucuguugccugugaggagcuuucuggcaugugauuauuuacuucaaaacuagaguuccaagcaccuacauuaauuauuuuauauugugugcagaauaguauaucuuuuaaugucagauaugauacacugcacauauugcuuuugcacucuuaaaauuuuuguacuaaauaauagaaaauauuuauauucuuugagugugagcuuugaauagauggcauuaucacuuuauuguuuuuuuaacaaaaacuuuuucucaauuauucuauugcaauguuauucugagcaaguccuaugccaaauaucuuguauaauguuuguauggaagauuaaauuuuacucuugugugguaagacuauuucaguuacugauuuuauaguuggaauuugauauuccagcacaaaguccacaguguauucagaaauccaaguuggugucauacauuucauuuugaugugaacuuuucuuugcuuuccuuuguucuaagacuccauuuugcaauaaacguuuugacaguaaaaaaaaaaaaa

SEQ ID NO: 3 - protein, 800 aa, Homo sapiens
MSSLGASFVQIKFDDLQFFENCGGGSFGSVYRAKWISQDKEVAVKKLLKIEKEAEILSVLSHRNIIQFYGVILEPPNYGIVTEYASLGSLYDYINSNRSEEMDMDHIMTWATDVAKGMHYLHMEAPVKVIHRDLKSRNVVIAADGVLKICDFGASRFHNHTTHMSLVGTFPWMAPEVIQSLPVSETCDTYSYGVVLWEMLTREVPFKGLEGLQVAWLVVEKNERLTIPSSCPRSFAELLHQCWEADAKKRPSFKQIISILESMSNDTSLPDKCNSFLHNKAEWRCEIEATLERLKKLERDLSFKEQELKERERRLKMWEQKLTEQSNTPLLPSFEIGAWTEDDVYCWVQQLVRKGDSSAEMSVYASLFKENNITGKRLLLLEEEDLKDMGIVSKGHIIHFKSAIEKLTHDYINLFHFPPLIKDSGGEPEENEEKIVNLELVFGFHLKPGTGPQDCKWKMYMEMDGDEIAITYIKDVTFNTNLPDAEILKMTKPPFVMEKWIVGIAKSQTVECTVTYESDVRTPKSTKHVHSIQWSRTKPQDEVKAVQLAIQTLFTNSDGNPGSRSDSSADCQWLDTLRMRQIASNTSLQRSQSNPILGSPFFSHFDGQDSYAAAVRRPQVPIKYQQITPVNQSRSSSPTQYGLTKNFSSLHLNSRDSGFSSGNTDTSSERGRYSDRSRNKYGRGSISLNSSPRGRYSGKSQHSTPSRGRYPGKFYRVSQSALNPHQSPDFKRSPRDLHQPNTIPGMPLHPETDSRASEEDSKVSEGGWTKVEYRKKPHRPSPAKTNKERARGDHRGWRNF

SEQ ID NO: 4 - protein, 455 aa, Homo sapiens
MSSLGASFVQIKFDDLQFFENCGGGSFGSVYRAKWISQDKEVAVKKLLKIEKEAEILSVLSHRNIIQFYGVILEPPNYGIVTEYASLGSLYDYINSNRSEEMDMDHIMTWATDVAKGMHYLHMEAPVKVIHRDLKSRNVVIAADGVLKICDFGASRFHNHTTHMSLVGTFPWMAPEVIQSLPVSETCDTYSYGVVLWEMLTREVPFKGLEGLQVAWLVVEKNERLTIPSSCPRSFAELLHQCWEADAKKRPSFKQIISILESMSNDTSLPDKCNSFLHNKAEWRCEIEATLERLKKLERDLSFKEQELKERERRLKMWEQKLTEQSNTPLLLPLAARMSEESYFESKTEESNSAEMSCQITATSNGEGHGMNPSLQAMMLMGFGDIFSMNKAGAVMHSGMQINMQAKQNSSKTTSKRRGKKVNMALGFSDFDLSEGDDDDDDDGEEEDNDMDNSE

SEQ ID NO: 5 - DNA, 22 bp, Homo sapiens
acacacatgtccttggttggaa

SEQ ID NO: 6 - DNA, 24 bp, Homo sapiens
tgacacagggagactctggataac

SEQ ID NO: 7 - DNA, 19 bp, Homo sapiens
cacctggctgggaaaatgg

SEQ ID NO: 8 - DNA, 19 bp, Homo sapiens
ggagcccttgtcggatgat